SEQUENCE LISTING (11 sequences)

SEQ ID NO: 1 - protein, 23 aa, Homo sapiens
MAPRGFSCLLLTSEIDLPVKRRA

SEQ ID NO: 2 - protein, 24 aa, synthetic construct
MAPRGFSCLLLLTGEIDLPVKRRA

SEQ ID NO: 3 - protein, 24 aa, synthetic construct
MAPRGFSALLLLTSEIDLPVKRRA

SEQ ID NO: 4 - protein, 24 aa, synthetic construct
MAAGAFSCLLLLTSEIDLPVKRRA

SEQ ID NO: 5 - protein, 17 aa, synthetic construct
PRGFSCLLLLTSEIDLP

SEQ ID NO: 6 - protein, 17 aa, synthetic construct
PRGFSCLLLLTGEIDLP

SEQ ID NO: 7 - protein, 17 aa, synthetic construct
PAGASRLLLLTGEIDLP

SEQ ID NO: 8 - protein, 26 aa, synthetic construct
SALLRSIPAPAGASRLLLLTGEIDLP

SEQ ID NO: 9 - protein, 11 aa, synthetic construct
CLLLTSEIDLP

SEQ ID NO: 10 - protein, 5 aa, synthetic construct
LLLLT

SEQ ID NO: 11 - protein, 5 aa, synthetic construct
EIDLP

(19) United States
(12) Patent Application Publication
(10) Pub. No.: US 2026/0166121 A1
(43) Pub. Date: Jun. 18, 2026

(54) COMPOSITIONS AND METHODS FOR THE TREATMENT OF HEMORRHAGIC SHOCK

(71) Applicant: Children's Hospital Medical Center, Cincinnati, OH (US)

(72) Inventor: Basilia Zingarelli, Cincinnati, OH (US)

(21) Appl. No.: 18/869,043

(22) PCT Filed: Jun. 2, 2023
(86) PCT No.: PCT/US2023/024273

§ 371 Date: Nov. 25, 2024

Related U.S. Application Data
(60) Provisional application No. 63/348,553, filed on Jun. 3, 2022.

(51) Int. Cl. A61K 38/17; A61K 45/06; A61P 7/04; G01N 33/68

(52) U.S. Cl. CPC A61K 38/1709; A61K 45/06; A61P 7/04; G01N 33/6869; G01N 2333/522; G01N 2333/525; G01N 2333/5412; G01N 2333/5428; G01N 2800/22; G01N 2800/52; G01N 2800/56

(57) ABSTRACT

Disclosed are methods of treating hemorrhagic shock, for example hemorrhagic shock associated with traumatic injury, gastrointestinal bleeding, spontaneous hemorrhage due to hematologic disorders, uterine hemorrhage, and combinations thereof, in an individual in need thereof. The methods may comprise administering a humanin protein or an analog thereof, for example Humanin G, to the individual.

### CROSS-REFERENCE TO RELATED APPLICATIONS

This application claims priority to and benefit of U.S. Provisional Application Ser. No. 63/348,553, filed Jun. 3, 2022, the contents of which are incorporated in their entirety for all purposes.

### STATEMENT REGARDING FEDERALLY-SPONSORED RESEARCH

This invention was made with government support under GM115973 and GM067202 awarded by the National Institutes of Health. The government has certain rights in the invention.

### BACKGROUND

Hemorrhagic shock is the leading cause of trauma-related deaths in patients surviving to the trauma center, accounting for about 40% of trauma deaths globally. The main therapeutic intervention following hemorrhage is to minimize the duration of tissue ischemia and preserve vulnerable tissue by bleeding control and restoration of adequate perfusion pressure. However, these interventions are not sufficient to prevent the activation of the systemic inflammatory cascade, which leads to multiple organ dysfunction syndrome (MODS) and eventually to the death of the patient. The exact mechanism of MODS following trauma is not known. There is a need for improved treatments for hemorrhagic shock. The instant disclosure seeks to address such need in the art.

### BRIEF SUMMARY

Disclosed are methods of treating hemorrhagic shock, for example hemorrhagic shock associated with traumatic injury, gastrointestinal bleeding, spontaneous hemorrhage due to hematologic disorders, uterine hemorrhage, and combinations thereof, in an individual in need thereof. The methods may comprise administering a humanin protein or an analog thereof, for example Humanin G, to the individual.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Effect of in vivo treatment with humanin-G on mean arterial blood pressure (A) and heart rate (B) in AMPKα1 wild-type (WT) and knockout (KO) mice. Vehicle (500 μl distilled water) or Humanin-G (100 μg/kg) was administered intra-arterially at the time of resuscitation and 1 h and 2 h thereafter. Each data represents the mean±SEM of 18-22 animals for each group. *Represents P<0.05 versus vehicle-treated AMPKα1-WT group; ‡represents versus Humanin G-treated AMPKα1-KO group; #represents P<0.05 versus vehicle-treated group of the same genotype.

FIG. 2. Plasma levels of humanin in AMPKα1 wild-type (WT) and knockout (KO) mice. Vehicle (500 μl distilled water) or Humanin-G (100 μg/kg) was administered intra-arterially at the time of resuscitation and 1 h and 2 h thereafter. Each data represents the mean±SEM of 12-17 for each group. *Represents P<0.05 versus control group of the same genotype.

FIG. 3. Representative histology photomicrographs of lung sections. Normal lung architecture in (A) control AMPKα1 wild-type (WT) and (D) knockout (KO) mice showing patent alveoli, and vessels with a few or no adhering neutrophils. Lung damage in vehicle-treated (B) WT and (E) KO mice after hemorrhagic shock with severe reduction of alveolar space, neutrophil adhesion along vascular walls, and infiltration of inflammatory cells. Amelioration of lung architecture in Humanin G-treated WT (C) and KO (F). Magnification 400×. A similar pattern was seen in n=4-6 different tissue sections in each experimental group. (G) Lung myeloperoxidase activity as index of neutrophil infiltration. Vehicle (500 μl distilled water) or Humanin-G (100 μg/kg) was administered intra-arterially at the time of resuscitation and 1 h and 2 h thereafter. Each data represents the mean±SEM of 7-12 animals for each group. *Represents P<0.05 versus control group of the same genotype; #represents P<0.05 versus vehicle-treated group of the same genotype.

FIG. 4. Plasma levels of cytokines in AMPKα1 wild-type (WT) and knockout (KO) mice. Plasma levels of IL-1β (A), IL-6 (B), IL-10 (C), IL-17 (D), KC (E), and TNFα (F). Vehicle (500 μl distilled water) or Humanin-G (100 μg/kg) was administered intra-arterially at the time of resuscitation and 1 h and 2 h thereafter. Each data represents the mean±SEM of 6-13 animals for each group. *Represents P<0.05 versus control group of the same genotype; ‡represents P<0.05 versus AMPKα1-WT group.

FIG. 5. Effect of in vivo treatment with humanin-G on cardiac mitochondrial damage and ATP levels. Transmission electron microscopy sections of heart tissue. Normal cellular structure was observed in control (A) AMPKα1 wild-type (WT) and (D) knockout (KO) mice with normal electron dense mitochondria presenting organized cristae. No abnormal structural changes were observed in vehicle-treated or humanin-G-treated WT mice (B and C) after hemorrhagic shock. In vehicle-treated KO mice (E) mitochondria presented with disrupted membrane and cristae, and translucent matrix (arrows). Amelioration of mitochondrial damage was observed in humanin-G treated KO mice (F) after hemorrhagic shock. Quantification of average mitochondrial area and damaged mitochondria (G) in myocytes as determined by using the NIH Image J software. Damaged mitochondria were determined as percentage of total number of mitochondria. ATP levels in heart tissue (H). Vehicle (500 μl distilled water) or Humanin-G (100 μg/kg) was administered intra-arterially at the time of resuscitation and 1 h and 2 h thereafter. Data are expressed as means±SEM of 3-4 animals for each group. *Represents P<0.05 versus control group of the same genotype; #represents P<0.05 versus vehicle-treated group of the same genotype; ‡represents P<0.05 versus AMPKα1-WT group.

FIG. 6. Representative Western blots of total STAT3, p-STAT3(Ser727) and p-STAT3(Tyr705) in cytosol and nuclear extracts of lung tissue. β-actin was used as loading control protein (A). Image analyses of cytosol and nuclear of relative intensity of ratio of p-STAT3(Ser727)/STAT3 (B), and ratio of p-STAT3(Tyr705)/STAT3 (C) as determined by densitometry. Vehicle (500 μl distilled water) or Humanin-G (100 μg/kg) was administered intra-arterially at the time of resuscitation and 1 h and 2 h thereafter. Each data represents the mean±SEM of 4-7 animals for each group. *Represents P<0.05 versus control group of the same genotype; #represents P<0.05 versus vehicle-treated group of the same genotype.

FIG. 7. Representative Western blots of total STAT3, p-STAT3(Ser727) and p-STAT3(Tyr705) in cytosol and nuclear extracts of heart tissue. β-actin was used as loading control protein (A). Image analyses of cytosol and nuclear of relative intensity of ratio of p-STAT3(Ser727)/STAT3 (B), and ratio of p-STAT3(Tyr705)/STAT3 (C) as determined by densitometry. Vehicle (500 μl distilled water) or Humanin-G (100 μg/kg) was administered intra-arterially at the time of resuscitation and 1 h and 2 h thereafter. Each data represents the mean±SEM of 4-7 animals for each group. *Represents P<0.05 versus control group of the same genotype.

FIG. 8. Representative Western blots of p-STAT3(Ser727), total STAT3, and GRIM-19 in mitochondrial extracts of lung tissue. VDAC-1 was used as loading control protein (A). Image analyses of relative intensity of ratio of p-STAT3(Ser727)/STAT3 (B), and GRIM-19 (C) as determined by densitometry. Vehicle (500 μl distilled water) or Humanin-G (100 μg/kg) was administered intra-arterially at the time of resuscitation and 1 h and 2 h thereafter. Each data represents the mean±SEM of 4-7 animals for each group. *Represents P<0.05 versus control group of the same genotype; ‡represents P<0.05 versus AMPKα1-WT group.

FIG. 9. Representative Western blots of p-STAT3(Ser727), total STAT3, and GRIM-19 in mitochondrial extracts of heart tissue. VDAC-1 was used as loading control protein (A). Image analyses of relative intensity of ratio of p-STAT3(Ser727)/STAT3 (B), and GRIM-19 (C) as determined by densitometry. Vehicle (500 μl distilled water) or Humanin-G (100 μg/kg) was administered intra-arterially at the time of resuscitation and 1 h and 2 h thereafter. Each data represents the mean±SEM of 4-7 animals for each group.

FIG. 10. Effect of genetic deficiency of AMPKα1 and HNG treatment on long-term survival. Kaplan-Meier survival curves were generated for 7 animals for each group. *Represents versus AMPKα1-WT group; #represents P<0.05 versus vehicle-treated group of the same genotype.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the treatment of hemorrhagic shock in an individual in need thereof.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “therapeutically effective amount” or “effective amount” mean the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to children.

The peptides and methods hereof may also comprise administering pro-drugs that metabolize to an active form of these peptides. As used herein, a “pro-drug” is a compound that a biological system metabolizes to an active compound as a result of spontaneous chemical reaction(s), enzyme catalyzed reaction(s), and/or metabolic chemical reaction(s), or a combination of each. Exemplary prodrugs may be formed using groups attached to functionality, e.g. HO—, HS—, HOOC—, R2N—, associated with the drug, that cleave in vivo. Further exemplary prodrugs include, but are not limited to, carboxylate esters where the group is alkyl, aryl, aralkyl, acyloxyalkyl, alkoxycarbonyloxyalkyl as well as esters of hydroxyl, thiol and amines, where the group attached is an acyl group, an alkoxycarbonyl, aminocarbonyl, phosphate or sulfate. The groups illustrated are exemplary and not exhaustive, and the present disclosure includes other known varieties of prodrugs.

“Sequence identity” as used herein indicates a nucleic acid or peptide sequence that has the same sequence as a reference sequence or has a specified percentage of nucleotides or amino acids that are the same at the corresponding location within a reference sequence when the two sequences are optimally aligned. For example a nucleic acid or peptide sequence may have at least 50%, 60%, 70%, 75%, 80%, 85%, 90%, 95%, 96%, 97%, 98%, 99%, or 100% identity to the reference nucleic acid or peptide sequence. The length of comparison sequences will generally be at least 5 contiguous nucleotides, or amino acids, or at least 10, 11, 12, 13, 14, 15, 16, 17, 18, 19, 20, 21, 22, 23, 24, 25 or more contiguous nucleotides or amino acids.

Disclosed herein are methods of treating hemorrhagic shock in an individual in need thereof, comprising administering a humanin protein or an analog thereof to the individual. In one aspect, the hemorrhagic shock may be associated with traumatic injury, gastrointestinal bleeding, spontaneous hemorrhage due to hematologic disorders, uterine hemorrhage, and combinations thereof. In other aspects, the humanin or humanin analog thereof may be administered for the treatment of organ injury consequent to ischemia and reperfusion injury conditions.

In one aspect, the humanin protein or analog thereof may comprise a peptide having from about 75% sequence identity, or about 80% sequence identity, or about 85% sequence identity, or about 90% sequence identity, or about 95% sequence identity to a sequence of Table 1. In one aspect, the humanin protein or analog thereof may have at least 90%, or at least 95% sequence homology to a peptide having a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In one aspect, the humanin protein or analog thereof may comprise a peptide having from about 75% sequence identity, or about 80% sequence identity, or about 85% sequence identity, or about 90% sequence identity, or about 95% sequence identity to a sequence of Table 1, wherein said protein or analog thereof comprises a sequence selected from CLLLTSEIDLP (SEQ ID NO: 9), LLLLT (SEQ ID NO: 10), EIDLP (SEQ ID NO: 11), or combinations thereof. In one aspect, the humanin protein or analog thereof may be selected from HN, HNG, HNA, AGA-HNG, HN17, HNG17, AGA-C8R-HNG17, colivelin, and combinations thereof.

TABLE 1
Humanin derivatives and colivelin
(Source: Matsuoka M. et al, Humanin and Colivelin: Neuronal-Death-
Suppressing Peptides for Alzheimer's Disease and Amyotrophic Lateral
Sclerosis, CNS Drug Reviews, Vol. 12, No. 2, pp. 113-122 (2006).)

| Name | Sequence | SEQ ID NO |
|---|---|---|
| Humanin (HN) | MAPRGFSCLLLTSEIDLPVKRRA | SEQ ID NO: 1 |
| Humanin G (HNG) | MAPRGFSCLLLLTGEIDLPVKRRA | SEQ ID NO: 2 |
| HNA | MAPRGFSALLLLTSEIDLPVKRRA | SEQ ID NO: 3 |
| AGA-HNG | MAAGAFSCLLLLTSEIDLPVKRRA | SEQ ID NO: 4 |
| HN17 | PRGFSCLLLLTSEIDLP | SEQ ID NO: 5 |
| HNG17 | PRGFSCLLLLTGEIDLP | SEQ ID NO: 6 |
| AGA-C84-HNG17 | PAGASRLLLLTGEIDLP | SEQ ID NO: 7 |
| Colivelin | SALLRSIPAPAGASRLLLLTGEIDLP | SEQ ID NO: 8 |

In one aspect, the humanin protein or analog thereof may be Humanin G (HNG), a synthetic mitochondrial peptide having the sequence MAPRGFSCLLLLTGEIDLPVKRRA (SEQ ID NO: 2).

In one aspect, the humanin protein or analog thereof may be modified. In one aspect, the modification may be that the humanin protein or analog thereof is PEGylated. In other aspects, the humanin protein or analog thereof may be administered as a pro-drug. In certain aspects, the humanin protein or analog thereof may be produced synthetically, and may, for example, comprise modified amino acids and/or D-amino acids which correspond to the L-amino acids of the listed sequences.

In one aspect, the humanin protein or analog thereof may be administered before, after, or simultaneously with a therapeutic amount of one or both of blood and fluid. In certain aspects, the humanin protein or analog thereof may be added to a fluid or blood source which is then administered to the individual in need thereof.

The humanin protein or analog thereof may be administered in an amount and duration as determined by the treating physician. For example, the amount and duration may be sufficient to improve lung injury, abnormal mean arterial blood pressure, survival, or combinations thereof in the individual. In other aspects, the humanin protein or analog thereof may be administered in an amount and duration sufficient to improve lung function. In a further aspect, the humanin protein or analog thereof may be administered in an amount and duration sufficient to ameliorate cardiac mitochondrial function associated with shock. In a further aspect, the humanin protein or analog thereof may be administered in an amount and duration sufficient to reduce Myeloperoxidase (MPO) activity. In further aspects, the administration of humanin or humanin analog, for example colivelin, may be administered sufficient to reduce oxidative damage in the small intestine and to reduce lung inflammation after mesenteric infarct.

Administration of the humanin protein or analog thereof may take a variety of forms. For example, the humanin protein or analog thereof may be administered via intramuscular (IM) delivery, intravenous (IV) delivery, subcutaneous (SC) delivery, intra-arterial delivery, oral delivery, gavage delivery, emollient/skin delivery, transdermal patch, and/or intranasally. The humanin protein or analog thereof may be administered continuously or as a dose. Exemplary dosing includes administering for at a period of at least one hour, or at least one day, or at least two days, or at least three days, or at least four days, or at least five days, or at least six days, or at least one week. The humanin protein or analog thereof may be administered within two hours of admission or diagnosis, within three hours of admission or diagnosis, within four hours of admission or diagnosis, within five hours of admission or diagnosis, or within six hours of admission or diagnosis.

Administration of the humanin protein or analog thereof may include administration of an amount of from about 50 μg/kg to 1000 μg/kg, from about 100 μg/kg to about 800 μg/kg, from about 200 μg/kg to about 600 μg/kg, or from about 300 μg/kg to about 500 μg/kg.

In one aspect, the individual may be a female. In one aspect, the individual may be a male.

In a further aspect, disclosed are methods for assessing one or both of a risk of lung injury and hemodynamic decompensation due to hemorrhagic shock. In this aspect, the methods may comprise detecting circulating humanin in the individual, wherein an increase in circulating humanin indicates that said individual has a higher risk of one or both of lung injury and hemodynamic decompensation.

In a further aspect, disclosed are methods for treating an individual for hemorrhagic shock. In this aspect, the method may comprise detecting a biomarker selected from IL-6, IL-10, KC, TNFα, and combinations thereof, and treating said individual having an elevation of said biomarker with humanin protein or an analog thereof to said individual, as disclosed herein. The hemorrhagic shock may be associated with traumatic injury, gastrointestinal bleeding, spontaneous hemorrhage due to hematologic disorders, uterine hemorrhage, and combinations thereof.

In a further aspect, disclosed are methods for monitoring the progression, severity, and/or likelihood of an individual having hemorrhagic shock ho is likely to progress to multiple organ dysfunction syndrome (MODS). In this aspect, the method may comprise detecting serum or blood levels of humanin in an individual at risk for multiple organ dysfunction syndrome, and treating the individual determined to have an increased risk of progressing to MODS with humanin, or an analog thereof, as described herein.

### Pharmaceutical Compositions

In one aspect, active agents provided herein may be administered in a dosage form selected from parenteral injection, continuous injection, oral administration, nasal administration, ophthalmic administration, buccal administration, and transdermal administration. In some aspects, the peptides provided herein may be formulated into liquid preparations for, e.g., oral administration.

In one aspect, the peptide-containing compositions may be isotonic with the blood or other body fluid of the recipient. The isotonicity of the compositions may be attained using, for example, sodium tartrate, propylene glycol or other inorganic or organic solutes such as sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

A pharmaceutically acceptable preservative may be employed to increase the shelf life of the pharmaceutical compositions, such as benzyl alcohol, parabens, thimerosal, chlorobutanol, or benzalkonium chloride. Preservatives may be added in an amount of from about 0.02% to about 2% based on the total weight of the composition, although larger or smaller amounts may be desirable depending upon the agent selected. Reducing agents, as described above, may be advantageously used to maintain good shelf life of the formulation.

In one aspect, the peptides provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and thus may be chosen according to the intended application, such that the characteristics of the carrier are tailored to the selected route of administration.

For oral administration of a peptide, the pharmaceutical compositions may be provided via a lipid-based nanocarrier such as, for example, one or more of oil-in-water nanoemulsions, self-emulsifying drug delivery systems (SEDDS), solid lipid nanoparticles (SLN), nanostructured lipid carriers (NLC), or liposomes and micelles.

In some aspects, the amount of peptide to be delivered or contained within a unit dose may be from about 1 mg or less to about 1,000 mg or more of a active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. A dosage appropriate to the patient and the number of doses to be administered daily may be selected. In certain aspects, two or more therapeutic agents (such as a peptide and antibiotic) may be incorporated to be administered into a single dosage form (e.g., in a combination therapy); however, in other aspects, the therapeutic agents may be provided in separate dosage forms.

In one aspect, the peptide is administered via injection. The duration of the therapy may be adjusted depending upon various factors, and may comprise a single injection administered daily, or twice a day, or three times a day, or over a longer period of time, such as every other day, every two days, every three days, every four days, every five days, every six days, every seven days, or weekly, every two weeks, every three weeks, or monthly. In other aspects, the peptide may be administered via continuous intravenous administration.

In some aspects, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of an active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Protective Effects of Humanin-G in Hemorrhagic Shock in Female Mice Via AMPKα1-Independent Mechanisms

Despite therapeutic advances in hemorrhagic shock, mortality from multiple organ failure remains high. The al subunit of AMP-activated protein kinase (AMPK), a crucial regulator of mitochondrial function, exerts a protective role in hemorrhagic shock. Humanin is a mitochondrial peptide with cytoprotective properties against cellular stress. Whether AMPKα1 influences systemic levels of endogenous humanin in hemorrhagic shock and whether treatment with the synthetic analogue humanin-G affords beneficial effects was investigated.

In brief, AMPKα1 wild-type (WT) and knock-out (KO) female mice were subjected to hemorrhagic shock followed by resuscitation with blood and Lactated Ringer's solution. In short-term studies mice were treated with humanin-G or vehicle and sacrificed at 3 hours after resuscitation; in survival studies, mice were treated with PEGylated humanin-G and monitored for 7 days. Compared to vehicle WT group, KO mice exhibited severe hypotension, cardiac mitochondrial damage, and higher plasma levels of Th17 cytokines but had similar lung injury and similar plasma elevation of endogenous humanin. Treatment with humanin-G improved lung injury, mean arterial blood pressure and survival in both WT and KO mice, without affecting systemic cytokine or humanin levels. Humanin-G also ameliorated cardiac mitochondrial damage and increased ATP levels in KO mice. Beneficial effects of humanin-G were associated with lung cytoplasmic and nuclear activation of the signal transducer and activator of transcription-3 (STAT3) in AMPKα1-independent manner with marginal or no effects on mitochondrial STAT3 and Complex I subunit GRIM-19. Thus, the data indicate that circulating levels of humanin increase during hemorrhagic shock in AMPKα1-independent fashion as a defense mechanism to counteract metabolic derangement, and that administration of humanin-G affords beneficial effects through STAT-3 activation even in the absence of a functional AMPKα1.

Mitochondrial dysfunction and cellular inability to use oxygen, despite resuscitation efforts to maximize oxygen delivery, have been suggested in organ injury in critical illness. Adenosine monophosphate-activated protein kinase (AMPK) is an important sensor and regulator of cellular energy status by promoting mitochondrial biogenesis. This kinase consists of a catalytic α-subunit and two regulating subunits Q and y, which are allosterically activated by low levels of ATP and high levels of AMP during metabolic stress including decreased oxygen supply. Activation of the AMPK pathway has an important role in favoring metabolic recovery in injured organs and reducing systemic inflammatory response, whereas genetic deficiency of the catalytic subunit AMPKα1 enhances organ injury and worsens hemodynamic instability during severe hemorrhagic shock in mice.

Humanin is a mitochondria-derived peptide, encoded by short open reading frame in the mitochondrial DNA, which has been described to have biological effects (Hashimoto Y, et al.: A rescue factor abolishing neuronal cell death by a wide spectrum of familial Alzheimer's disease genes and Abeta. Proc Natl Acad Sci USA 98:6336-6341, 2001.). Like hormones, the peptide can be secreted extracellularly and is found both in tissues and circulation of rodents and humans. Originally isolated from a cDNA library of surviving neurons of familial Alzheimer's disease, humanin has been shown neuroprotective effects in rodent models of neurodegenerative disorders and to reduce infarct size in cerebral ischemia and reperfusion injury. In vitro studies have demonstrated that humanin and synthetic humanin analogues protect against cell death and oxidative stress by involving activation of the signal transducer and activator of transcription-3 (STAT3). STAT3 is a crucial transcription factor, which plays a role in development, inflammation, immunity, metabolism and cancer. In addition to its established role as a nuclear transcription factor, a pool of STAT3 has been described in the mitochondria, where it regulates Complex I activity and enhances ATP production. Despite the substantial literature on humanin-mediated beneficial effects in neurological diseases, the effect of humanin on organ injury during the oxidative stress of hemorrhagic shock has not been investigated.

By employing a clinically relevant model of hemorrhagic shock in female mice Applicant sought to evaluate the therapeutic efficacy of a potent synthetic humanin analogue, humanin-G, in which the serine of humanin at position 14 is replaced by glycine (Terashita K, et al.: Two serine residues distinctly regulate the rescue function of Humanin, an inhibiting factor of Alzheimer's disease-related neurotoxicity: functional potentiation by isomerization and dimerization. J Neurochem 85(6):1521-1538, 2003) and its potential molecular mechanisms of action on STAT3. Because of the central role of AMPK in mitochondrial quality control, whether AMPKα1 is involved in the endogenous production of humanin and whether it may contribute to potential therapeutic effects of the synthetic analogue humanin-G was evaluated.

Humanin-G Ameliorates Hemodynamic Instability in AMPKα1-Dependent and -Independent Manner after Hemorrhagic Shock

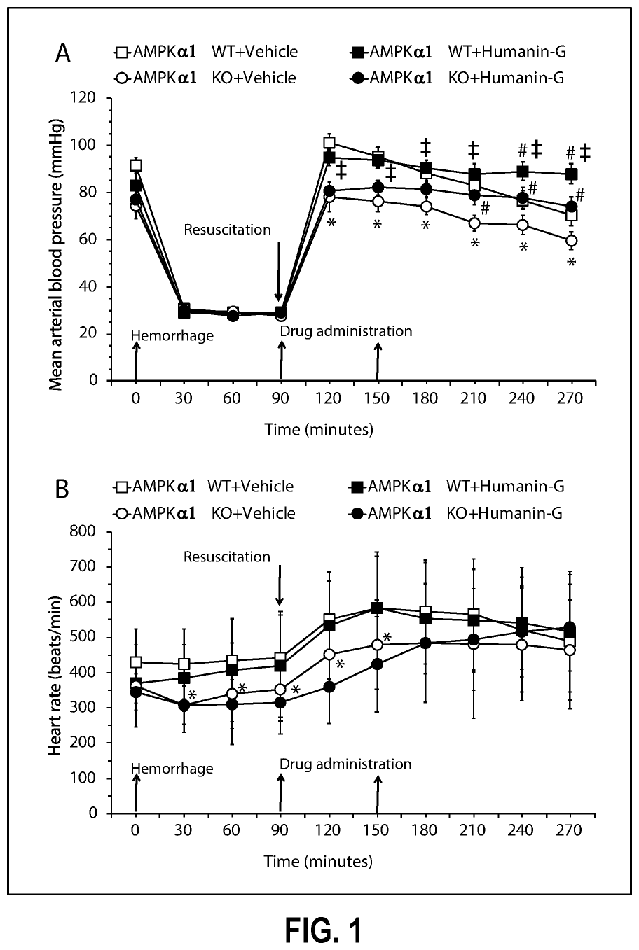

To maintain a similar degree of hypoperfusion, both AMPKα1 WT and KO mice were hemorrhaged to a MABP of 30±5 mmHg and were resuscitated at 90 minutes after the initial hemorrhage. After an early increase subsequent to transfusion, MABP progressively declined in both vehicle-treated groups; however, AMPKα1 KO animals experienced more severe hypotension compared to WT mice (FIG. 1, panel A). Treatment with humanin-G significantly ameliorated MABP in both AMPKα1 WT and KO animals compared to the vehicle-treated groups. However, in AMPKα1 KO mice MABP did not completely recover to baseline, suggesting that the mechanism of action of humanin-G is at least partially dependent on AMPKα1. Vehicle-treated AMPKα1 KO mice experienced relative bradycardia during hemorrhagic shock, but HR gradually recovered after resuscitation to similar levels as WT mice (FIG. 1, panel B). Humanin-G treatment did not affect HR in either AMPKα1 WT or KO mice when compared to vehicle-treated groups.

Plasma Levels of Humanin Increase in AMPKα1-Independent Manner after Hemorrhagic Shock

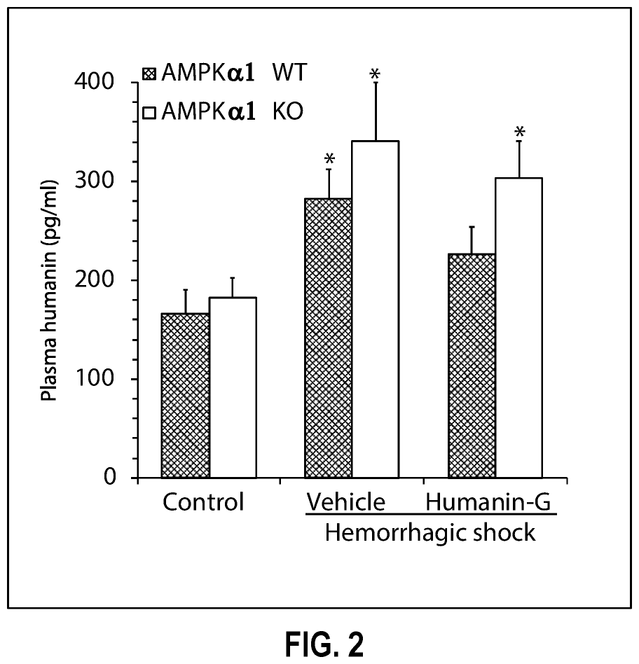

Applicant next determined whether hemodynamic instability is associated with changes in circulating levels of the endogenous humanin (FIG. 2). There was no difference in plasma levels of endogenous humanin at basal conditions between control AMPKα1 WT and KO mice. After hemorrhagic shock, AMPKα1 WT and KO mice had significantly higher plasma levels of humanin when compared to their genotype-matched control mice. Plasma humanin levels in humanin-G-treated AMPKα1 WT and KO mice were not statistically different compared to levels of vehicle-treated groups. However, in humanin-G-treated AMPKα1 WT mice, but not KO mice, plasma levels of humanin were comparable with levels of control mice, suggesting a decline towards basal physiological levels.

Humanin-G Ameliorates Lung Injury and Tissue Neutrophil Infiltration in AMPKα1-Independent Manner after Hemorrhagic Shock

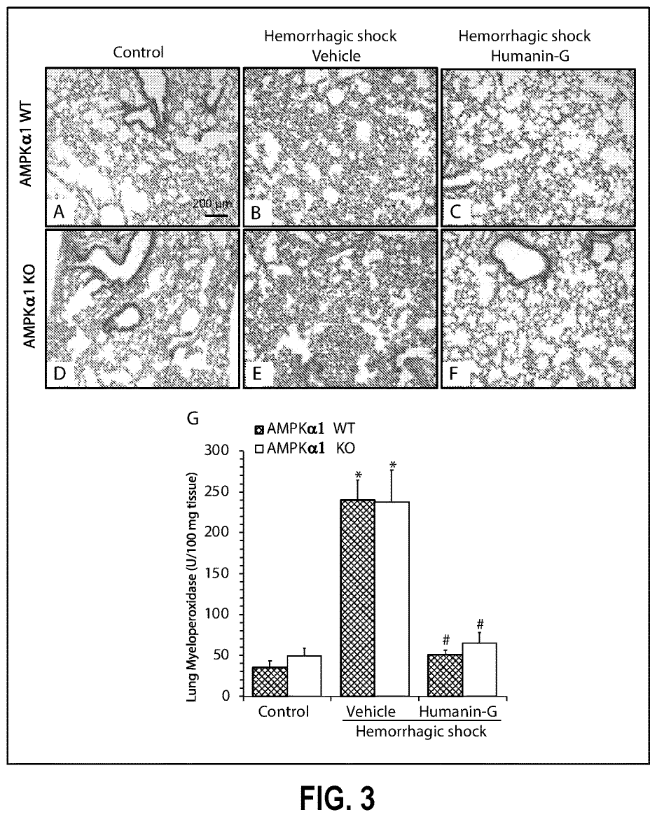

Despite the AMPKα1 KO mice having more hemodynamic instability, lung injury was similar between AMPKα1 WT and KO mice after hemorrhagic shock (FIG. 3). At baseline conditions, both AMPKα1 WT and KO mice exhibited similar normal lung architecture (FIGS. 3, panel A and 3, panel D). After hemorrhagic shock, lung injury was characterized by significant neutrophil margination and infiltration, atelectasis, alveolar disruption, proteinaceous debris, and hemorrhage in vehicle-treated mice of both genotypes (FIGS. 3, panel B and 3, panel E). Treatment with humanin-G significantly improved the lung architecture in both AMPKα1 WT and KO mice (FIGS. 3, panel C and 3, panel F). To further confirm the degree of neutrophil infiltration, activity of MPO, a lysosomal enzyme specific to neutrophils, was measured. MPO activity was similarly elevated after hemorrhagic shock in mice of both genotypes. Treatment with humanin-G significantly reduced MPO activity in both AMPKα1 WT and KO mice (FIG. 3, panel G).

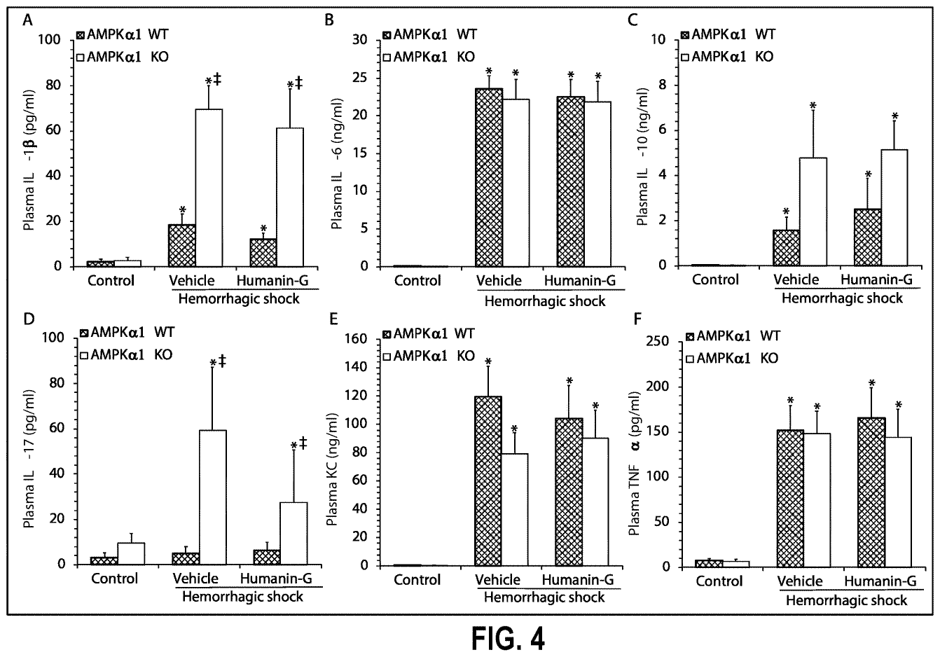

Humanin-G does not affect plasma levels of cytokines. To evaluate the effect of treatment with Humanin-G on systemic inflammation, a panel of Th1/Th2/Th17 cytokines was measured (FIG. 4). Following hemorrhagic shock, plasma levels of IL-1β and IL-17 were significantly higher in AMPKα1 KO mice when compared to WT mice. Plasma levels of IL-6, IL-10, KC, and TNFα were similarly elevated after hemorrhagic shock in mice of both genotypes. Treatment with humanin-G did not affect plasma levels of cytokines in either WT or KO mice.

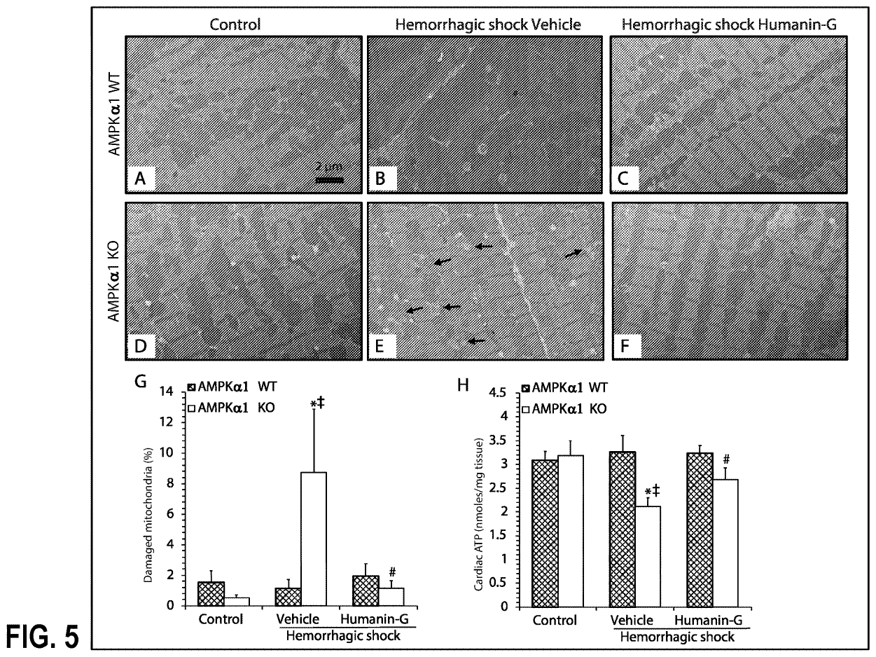

Humanin-G reduces cardiac cell mitochondrial damage and improves ATP production in AMPKα1 KO mice. Because cardiac cells are highly metabolically active, mitochondrial structure and function of cardiac tissue was evaluated by electron microscopy and by measurement of ATP (FIG. 5). At basal conditions, mitochondria of both AMPKα1 WT and KO control mice exhibited similar normal structure (FIGS. 5, panel A, and 5, panel D). After hemorrhagic shock, vehicle-treated AMPKα1 KO mice exhibited a higher percentage of damaged mitochondria with membrane loss and disorganized cristae when compared to WT mice (FIGS. 5, panel B, 5, panel C, and 5, panel F). ATP levels significantly decreased in vehicle-treated AMPKα1 KO mice, but not in the WT group, after hemorrhagic shock when compared to baseline values of genotype-matched control mice (FIG. 5, panel H). Treatment with humanin-G reduced mitochondrial damage and increased cardiac ATP levels in KO mice (FIG. 5).

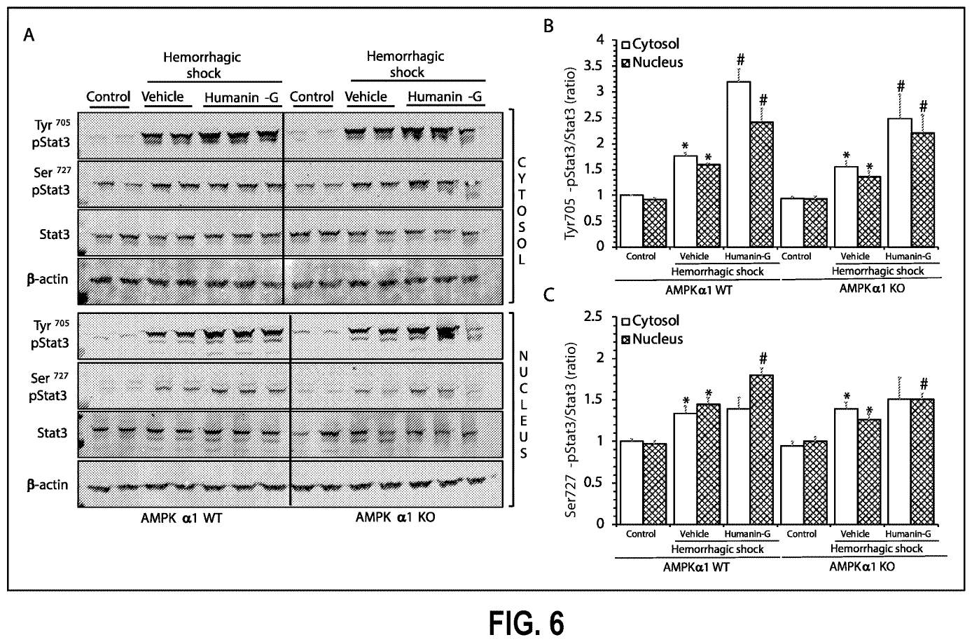

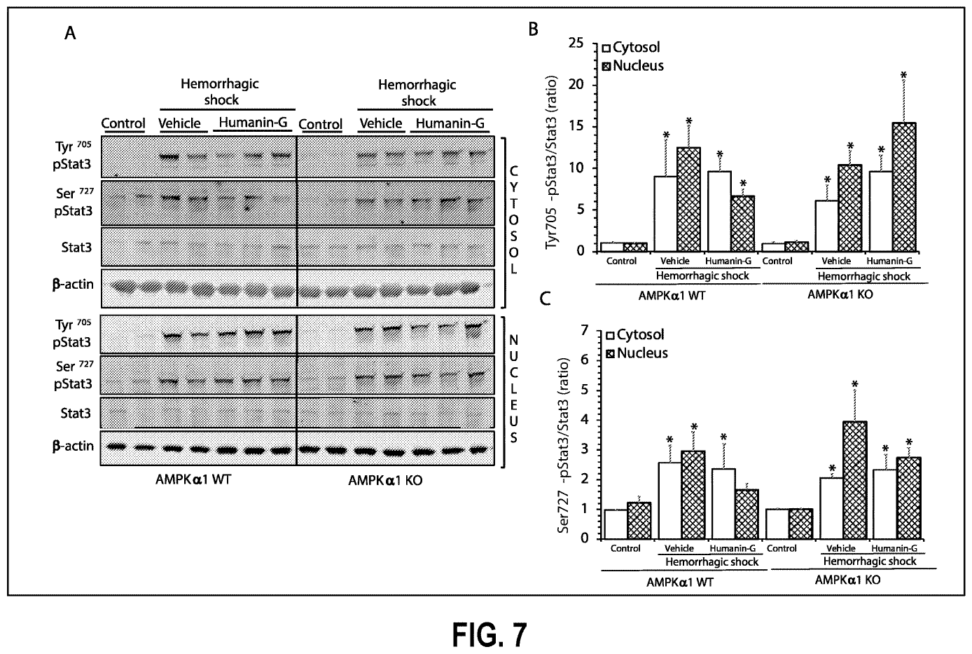

Humanin-G increases STAT3 activation in the lung, but not in the heart. Since humanin-G afforded beneficial effects in the absence of a functional AMPKα1 gene in KO mice, whether the molecular mechanisms of humanin-G are associated with changes in subcellular localization and activation of STAT3 in the lung and the heart were investigated. In the lung, control AMPKα1 WT and KO mice exhibited marginal levels of pSTAT3(Ser727) and pSTAT3(Tyr705) in both cytosol and nuclear compartments (FIG. 6). Following hemorrhagic shock both pSTAT3(Ser727) and pSTAT3(Tyr705) were upregulated in the cytosol and nucleus in mice of both genotypes when compared to basal levels of control mice, thus suggesting an overall activation of the transcription factor (FIG. 6). Following humanin-G administration, pSTAT3(Tyr705) was further upregulated in both cytosol and nucleus in mice of both genotypes, whereas pSTAT3(Ser727) was upregulated only in the nucleus. There was no difference in the total levels of STAT3 across the experimental groups in either WT or KO mice. In the heart, control AMPKα1 WT and KO mice exhibited very low expression of pSTAT3(Ser727) and pSTAT3(Tyr705) in both cytosol and nuclear compartments (FIG. 7). Following hemorrhagic shock both pSTAT3(Ser727) and pSTAT3(Tyr705) were upregulated in the cytosol and nucleus in mice of both genotypes when compared to basal levels of control mice, thus suggesting an overall activation of the transcription factor (FIG. 7). Treatment with humanin-G did not affect the cytosolic or nuclear expression of pSTAT3(Tyr705) or pSTAT3(Ser727) in WT mice. There was no difference in the total levels of STAT3 across the experimental groups in either WT or KO mice (FIG. 7).

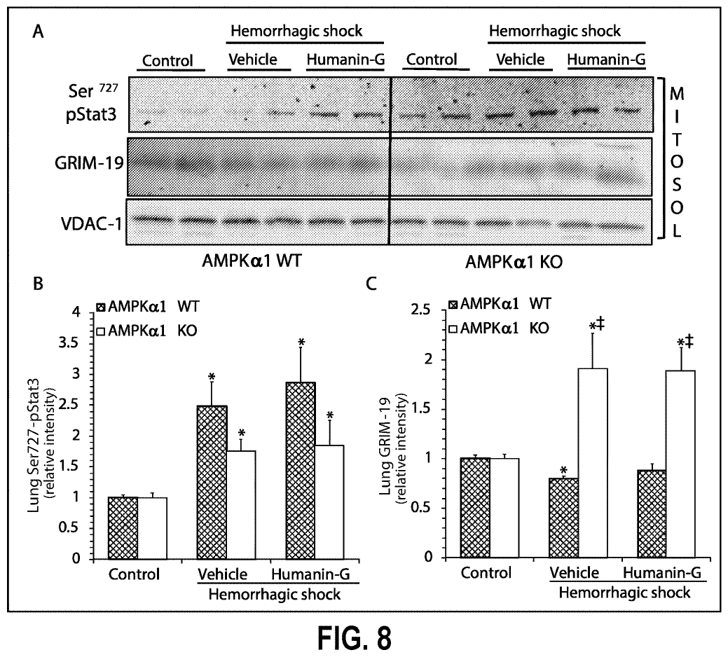

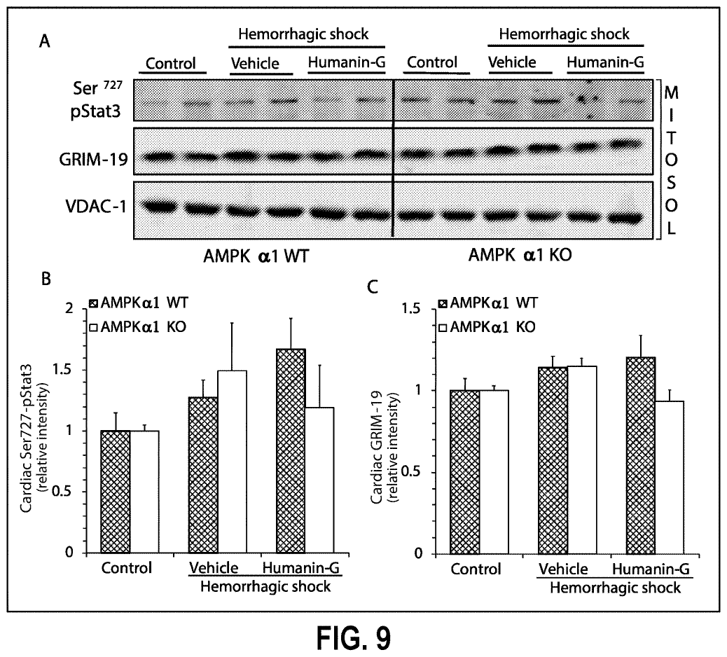

Effect of humanin-G treatment on mitochondrial pSTAT3(Ser727) and GRIM-19 expression in lung and heart. To better determine the intracellular activation of STAT3, whether pSTAT3(Ser727) also localizes in the mitochondria and whether it is associated with changes of the Complex-1 subunit GRIM-19 in lung and heart was investigated. In isolated lung mitochondria, pSTAT3(Ser727) expression was significantly increased in both AMPKα1 WT and KO animals following hemorrhagic shock when compared to basal levels of control mice (FIG. 8). Treatment with humanin-G slightly, but not significantly, increased the mitochondrial expression of pSTAT3(Ser727) in WT and KO mice. GRIM-19 expression decreased in vehicle-treated WT animals following hemorrhagic shock when compared to basal levels of control mice. On the contrary, GRIM-19 expression was significantly higher in vehicle-treated KO animals following hemorrhagic shock when compared to basal levels of KO control mice and to levels of vehicle-treated WT mice. Treatment with humanin-G restored levels of GRIM-19 similarly to basal levels in WT mice, whereas it did not affect GRIM-19 expression in KO mice. In the heart mitochondria, pSTAT3(Ser727) and GRIM-19 expression was slightly increased, but not significantly different, in both WT and KO animals following hemorrhagic shock when compared to basal levels of control mice (FIG. 9). Treatment with humanin-G did not affect mitochondrial expression of pSTAT3(Ser727) or GRIM-19 in either WT or KO mice when compared to vehicle-treated mice.

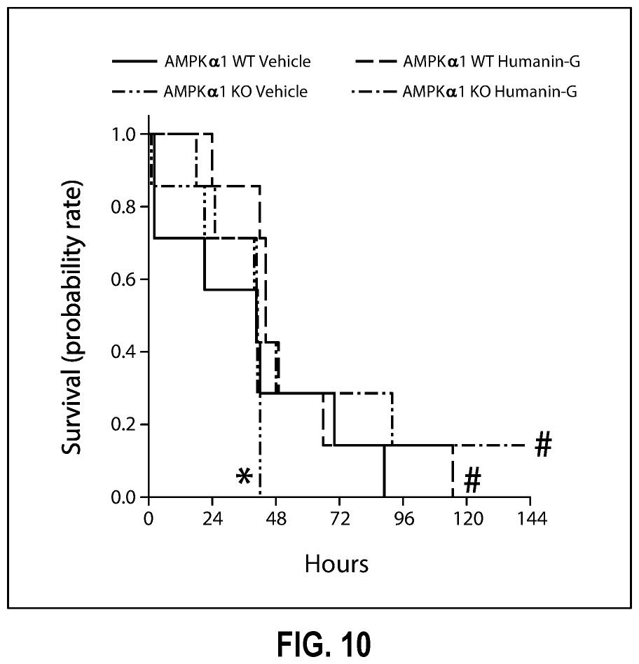

Treatment with PEGylated-humanin-G improves survival after hemorrhagic shock. To evaluate the long-term therapeutic potential of humanin-G, Applicant generated a PEGylated form of the peptide, and the effect of the drug on survival up to 7 days after hemorrhagic shock was tested (FIG. 10). Vehicle-treated AMPKα1 WT mice lived significantly longer than vehicle-treated KO mice. Treatment with PEGylated humanin-G significantly ameliorated survival in both AMPKα1 WT and KO mice when compared to vehicle treatment.

### Discussion

Applicant has demonstrated that increased plasma levels of the mitochondrial peptide humanin occurred in an AMPKα1-independent fashion at the early stage of hemorrhagic shock and coincided with hemodynamic instability and lung injury in female mice. It is also demonstrated for the first time that humanin-G, a potent synthetic humanin derivative, is a potential therapeutic peptide which improves outcomes of hemorrhagic shock. It was found, in fact, that in vivo humanin-G administration, when given as an adjunctive treatment to the standard blood and fluid resuscitation, afforded hemodynamic stability, attenuated lung injury, and improved long-term recovery. In the lung, but not in the heart, the mechanisms of action of humanin-G were AMPKα11-independent and involved the activation of the STAT3 pathway.

Despite advances in resuscitation, MODS remains a significant cause of morbidity and mortality following hemorrhagic shock. In addition to traumatic injury, severe acute bleeding may also be consequent to non-traumatic clinical conditions such as gastrointestinal bleeding, spontaneous hemorrhage in patients with hematologic disorders, and uterine hemorrhage in female patients. To mimic this latter condition of non-traumatic injury in female patients, in the present study an experimental model of fixed pressure-controlled hemorrhagic shock in female mice was adopted. Applicant investigated the pathogenetic mechanisms of organ injury with a focus on mitochondrial dysfunction, which has been proposed as a major contributing factor of MODS. AMPK is a pathway involved in maintaining energy metabolism under pathological processes of multiple organ failure in male mice since genetic inhibition of AMPKα1 or age-dependent dysfunction of the enzyme is associated with heightened severity of pulmonary and cardiac injury, and systemic inflammation after hemorrhagic shock. To confirm the biological role of AMPK in a female population, Applicant demonstrated that genetic inhibition of AMPKα1 also worsens hemodynamic decompensation in young female AMPKα1 KO mice and is associated with mitochondrial damage and impaired ATP production in the heart. Genetic inhibition of AMPKα1 was also associated with higher plasma levels of IL-1β and IL-17 in female KO mice when compared to WT mice after hemorrhagic shock. IL-1β is a proinflammatory cytokine that drives immune responses by activating T helper (Th)17 cells, which produce IL-17 among other cytokines. Several lines of evidence have documented that AMPK plays a decisive role in regulation of Th17 cell generation. It has been reported that AMPK activators could effectively inhibit Th17 differentiation, whereas the AMPK inhibitor Compound C could promote Th17 differentiation. Thus, Applicant found that AMPKα1 is an important regulator of Th17 responses in hemorrhagic shock. Despite a worse hemodynamic decompensation, genetic inhibition of AMPKα1 in KO female mice was not associated with differences in lung injury or plasma levels of cytokines of the Th1 or Th2 response when compared to WT mice. These findings are different from Applicant's previous studies demonstrating that male AMPKα1 KO mice exhibited higher levels of several inflammatory cytokines of the Th1 or Th2 response and higher lung injury when compared to WT male mice. Thus, the data suggest a significant effect of sex on outcomes of hemorrhagic shock. Treatment with humanin-G did not result in decreased cytokine production, thus suggesting that metabolic, rather than inflammatory, changes are responsible for its protective effects.

In addition to energy failure, mitochondrial dysfunction is intertwined with major perturbations in intracellular communication. Mitochondria-derived peptides, encoded by short open reading frame in the mitochondrial DNA, have been recently described to have biological effects. Humanin is a 24-amino acid peptide originally isolated from a neuronal cDNA library of patients with Alzheimer's disease. Several experimental studies describe potent cytoprotective effects of humanin and its analogues. For example, humanin is shown as a rescue factor against neuronal cell death and to protect endothelial cells from oxidative stress induced by oxidized LDL. Administration of humanin has also been reported to reduce infarct size and confer protection in rodent model of cerebral and myocardial ischemia and reperfusion. Increased systemic levels of humanin have been reported in pregnant women with pre-eclampsia when compared with women without pre-eclampsia and have been correlated with endothelial injury. Here, plasma humanin levels significantly rose after hemorrhagic shock in both AMPKα1 WT and KO young female mice when compared to physiological baseline values of control mice, thus suggesting that elevation of humanin levels may represent a defense response against oxidative stress. Interestingly, AMPKα1 did not appear to have a regulatory role in humanin release in circulation; AMPKα1 KO mice had similar circulating levels of humanin as WT mice.

Applicant found that in vivo treatment with the synthetic analog humanin-G, which shows stronger activity than the original peptide, improved hemodynamic compensation, organ injury and long-term outcomes of mice subjected to hemorrhagic shock. These data also support Applicant's recent findings demonstrating that colivelin, another synthetic analogue of humanin, exerts protective effects against organ injury in experimental septic shock (Urban C, et al.: Colivelin, a synthetic derivative of humanin, ameliorates endothelial injury and glycocalyx shedding after sepsis in mice. Front Immunol 13:984298, 2022). In the current study, the beneficial effects of the synthetic peptide were seen in both AMPKα1 WT and KO mice, thus suggesting, at least partially, AMPKα1-independent effects.

To investigate the mechanisms responsible for the beneficial effects of humanin-G, the contribution of the STAT3 signaling pathway was investigated. In vitro studies have, in fact, shown that treatment with humanin and its analogues may exert protective functions through STAT3 phosphorylation. STAT3 is a transcription factor, which is involved in many immunological and inflammatory functions and in the regulation of cell apoptosis. In addition to its cytosolic and nuclear location, STAT3 has been described in the mitochondria. STAT3 in the mitochondria requires Ser727 but not Tyr705 phosphorylation and it exerts metabolic effects through direct or indirect regulation of the electron transport chain for ATP production. One direct regulatory mechanism of STAT3 on mitochondrial respiration has been reported to occur after association with Complex I, most probably through regulation of GRIM-19. Applicant examined the subcellular localization of the different phosphorylated forms of STAT3, and observed for the first time that pSTAT3(Tyr705) and pSTAT3(Ser727) are activated in the cytosol and nuclear compartments of lung after hemorrhagic shock in vehicle-treated mice. Applicant found that treatment with humanin-G further increased cytosol and nuclear activation of STAT3 in the lung and was associated with amelioration of damage in an AMPKα1-independent fashion. These findings are consistent with previous studies reporting that increased activation of STAT3 prevents liver injury in rodent models of hemorrhagic shock. Applicant also found that a distinct localization of the pSTAT3(Ser727) in lung mitochondria, which increased after hemorrhagic shock in vehicle-treated mice of both genotypes, most probably suggesting a defense mechanism against oxidative stress in the mitochondria independent of the AMPKα1 pathway. This mitochondrial pool of STAT3 was only slightly, but not significantly, affected by the treatment with humanin-G, thus suggesting that the protective effects of the synthetic peptides are more likely to be mediated by several cytosolic and nuclear STAT3-dependent pathways. Expression pattern of pSTAT3(Ser727) did not parallel the expression of GRIM-19 since this Complex I subunit was down-regulated in vehicle-treated AMPKα1 WT mice whereas it was upregulated in AMPKα1 KO mice after hemorrhagic shock. Levels of GRIM-19 were restored to basal levels in AMPKα1 WT but not KO mice after treatment with humanin-G. It is difficult to determine the exact role of GRIM-19 in the heart in the context of oxidative stress. While GRIM-19 is important for maintenance of mitochondrial transmembrane potential, when GRIM-19 accumulates in excess into the mitochondria it may induce deleterious effects. For example, excessive mitochondrial accumulation of GRIM-19 has been associated with increase of reactive oxygen species generation and cytotoxicity in cancer cells. Increased GRIM-19 protein levels in neuronal mitochondria were found to correlate with increased oxidative stress in rat brain ischemia. Thus, the data suggest that AMPKα1 may regulate the degree to which GRIM-19 accumulates in the mitochondria, thus influencing the cell fate without affecting STAT3 phosphorylation.

The STAT3-dependent mechanisms of action of humanin-G appeared specific for the pulmonary protective effects since treatment with the synthetic peptide did not affect activation of STAT3 in any of the subcellular compartments of the heart. The synthetic mitochondrial peptide colivelin activates both AMPKα1 and AMPKα2 subunits in thoracic aortas thus ameliorating endothelial damage in mice with sepsis. Furthermore, mitochondrial peptides have been described to activate AMPK and to interact with several cell surface receptors, such as formylpeptide-like-1 receptor and insulin-like growth factor binding protein-3.

In conclusion, the data indicates that elevation of circulating humanin is an early event and parallels lung injury and hemodynamic decompensation in a murine model of hemorrhagic shock. Treatment with the synthetic mitochondrial peptide humanin-G exerts pulmonary protective effects and improves long-term recovery, at least in part, via activation of the STAT3 pathway in the lung. The data indicate that humanin and its analogues may be used to afford pulmonary protective effects and to ameliorate cardiac mitochondrial function in conditions of shock.

### Materials and Methods

Murine model of hemorrhagic shock. The experiments conformed to the Guide for the Care and Use of Laboratory Animals published by the US National Institutes of Health (Eighth edition, 2011) and had the approval of the Institutional Animal Care and Use Committee. Homozygous AMPKα1 wild-type (WT) and AMPKα1 knockout (KO) mice were established on a C57/BL6 genetic background by the crossbreeding (>10 generations) of a breeding pair provided by Dr. Benoit Viollet of the University of Paris Descartes (Paris, France). For these experiments, female AMPKα1 WT and KO mice were generated by a breeding scheme utilizing heterozygous mutant mice. Female mice were used at random stages of the estrous cycle at the age of 3 to 5 months and assigned to the different experimental groups after routine genotyping by quantitative polymerase chain reaction. All mice were allowed free access to water and a maintenance diet in a 12-h light/dark cycle, with room temperature at 21±2° C. Mice were anesthetized with pentobarbital (80 mg/kg) intraperitoneally and prepared for hemorrhagic shock, as previously described. Briefly, either the left or right femoral artery was cannulated using PE-10 tubing, and connected to a blood pressure transducer (PowerLab, AD Instruments, Colorado Springs, CO) for measurement of mean arterial blood pressure (MABP) and heart rate (HR). Blood was removed from the femoral artery over a 10-minute period until MABP reached 30±5 mmHg. The mice were kept in this MABP range for 90 min by additional blood removal or small volume transfusion. At the end of the shock period, mice were resuscitated by infusing their shed blood and twice that amount in Lactated Ringer's solution over a 10-min period. AMPKα1 WT and KO mice were also randomly assigned to two treatment groups: a vehicle group (distilled water, 500 μL) and a humanin-G (100 μg/kg) group. Drug treatment was given intra-arterially as single bolus right before resuscitation, and at 1 and 2 hours thereafter. Dose of humanin was chosen according to reported pharmacokinetics and pharmacodynamics of the drug in the mice. Mice were monitored for 3 hours after resuscitation for cardiovascular parameters. Control mice were anesthetized but did not undergo any surgical preparation or blood removal. Mice were sacrificed at 3 hours after resuscitation and blood, heart and lungs were collected for biochemical assays.

Plasma levels of humanin. Endogenous plasma levels of humanin were measured by competitive ELISA (Mouse Putative Mouse in Peptide, MyBioSource, San Diego, CA) using the protocol recommended by the manufacturer.

Plasma levels of cytokines. Plasma levels of interleukin (IL)-1β, IL-6, IL-10, IL-17, keratinocyte-derived chemokine (KC), and tumor necrosis factor-α (TNFα) were evaluated by a commercially available multiplex array system (Linco-Research, St. Charles, MO) using the protocol recommended by the manufacturer.

Histopathologic analysis. Lungs were fixed in 4% paraformaldehyde and embedded in paraffin. Sections were stained with hematoxylin and eosin and evaluated by an independent observer blinded to the treatment groups. Lung injury was based on the following histologic features: alveolar capillary congestion, infiltration of red blood and inflammatory cells into the airspace, alveolar wall thickness, and hyaline membrane formation.

Myeloperoxidase assay. Myeloperoxidase (MPO) activity was determined as an index of neutrophil accumulation. Lung samples were homogenized in a solution containing 0.5% hexa-decyl-trimethyl-ammonium bromide dissolved in 10 mM potassium phosphate buffer (pH 7.0) and centrifuged for 30 min at 4000×g at 4° C. An aliquot of the supernatant was allowed to react with a solution of tetra-methyl-benzidine (1.6 mM) and hydrogen peroxide (0.1 mM). The rate of change in absorbance was measured by spectrophotometry at 650 nm. MPO activity was defined as the quantity of enzyme degrading 1 mol of hydrogen peroxide/min at 37° C. and expressed in units per 100 mg weight of tissue.

Preparation of PEGylated humanin-G and survival analysis. In separate groups of mice survival studies were conducted. For these studies, we generated a longer-acting humanin-G by covalent conjugation of polyethylene glycol (PEG). Briefly, humanin-G was conjugated (1:1 molar ratio) to generate a non-cleavable PEGylated humanin-G. Humanin-G was dissolved in phosphate-buffered saline, pH 7.4, with 5 mM EDTA and mixed at room temperature with an equal volume of DMSO containing PEG-maleimide and left overnight (26). The product, PEGylated humanin-G (PEG10 kDa) was purified by size-exclusion chromatography with a 50 mM ammonium acetate buffer. Mice were subjected to hemorrhagic shock as described above. Mice received PEGylated humanin-G (100 μg/kg, intra-arterially) as a single dose at 90 minutes after blood removal at the time of resuscitation. Three hours after resuscitation, the femoral artery was occluded, and the skin incision was closed in layers. Mice were then allowed to awaken from anesthesia. Mice then received a second and a third dose of PEGylated humanin-G (100 μg/kg, subcutaneously) at 24 hours and 48 hours thereafter. Mice also received further fluid resuscitation (0.5 mL of Lactated Ringer's solution subcutaneously) at 6 hours and 24 hours following hemorrhagic shock. Analgesia was provided with buprenorphine (0.05 mg/kg, subcutaneously) immediately the surgical procedures and once daily for the first three days. Mice were then monitored for survival for 7 days.

Transmission electron microscopy. Heart tissue samples were fixed in 3% glutaraldehyde and postfixed in 1% osmium tetroxide in sodium phosphate buffer. Sections were stained with 2% uranyl acetate and lead citrate and were photographed on Hitachi H-7650 transmission electron microscope at 120 kV. The total number of mitochondria, and the presence of abnormal mitochondria with loose matrix, fragmented cristae and membranes were determined in four consecutive cells in four different sections for each animal by using NIH ImageJ analysis.

ATP assay. ATP levels were measured in heart tissue homogenates using a commercially available ATP assay kit (BioVision, Milpitas, CA) and following the protocol recommended by the manufacturer.

Cytosol and nuclear extracts. Heart and lung tissues were homogenized using a Polytron homogenizer (Brinkman Instruments, West Orange, NY) in a buffer containing 0.32 M sucrose, 10 mM TrisHCl (pH 7.4), 1 mM EGTA, 2 mM EDTA, 5 mM NaN3, 10 mM β-mercaptoethanol, 20 mM leupeptin, 0.15 mM pepstatin A, 0.2 mM phenylmethanesulfonyl fluoride, 50 mM NaF, 1 mM sodium orthovanadate, and 0.4 nM microcystin. Samples were centrifuged at 1,000 g for 10 min at 48° C. and the supernatants collected as cytosol extracts. The pellets were then solubilized in Triton buffer (1% Triton X-100, 250 mM NaCl, 50 mM Tris HCl at pH 7.5, 3 mM EGTA, 3 mM EDTA, 0.1 mM phenylmethanesulfonyl fluoride, 0. 1 mM sodium orthovanadate, 10% glycerol, 2 mM p-nitrophenyl phosphate, 0.5% NP-40, and 46 mM aprotinin). The lysates were centrifuged at 15,000 g for 30 min at 48° C. and the supernatant collected as nuclear extracts. The Bradford protein assay was used for quantitative determination of total proteins.

Mitochondrial extracts. Mitochondria were isolated from hearts and lungs using a commercially available mitochondria isolation kit (TermoFisher Scientific, Waltham, MA) and following the protocol recommended by the manufacturer. The Bradford protein assay was used for quantitative determination of total proteins.

Western blot analysis. Cytosol and nuclear content of STAT3 and its two phosphorylated active forms pSTAT3(Tyr705) and pSTAT3(Ser727), and mitochondrial content of pSTAT3(Ser727) and the Complex I subunit GRIM-19 (gene associated with retinoid-IFN-induced mortality) were determined by immunoblotting analyses. Extracts were heated at 70° C. in equal volumes of ×4 Protein Sample Loading Buffer. Twenty μg of protein were loaded per lane on a 10% Bis-Tris gel. Proteins were separated electrophoretically and transferred to nitrocellulose membranes. Immunoblotting was performed using the IBind Flex Western System (Thermo Fischer Scientific, Waltham, MA) that applies sequential lateral flow to perform blocking and antibody binding. Loading control proteins were concomitantly probed: 13-actin as loading control in cytosol and nuclear extracts; and voltage-dependent anion channel-1 (VDAC-1) as loading control in mitochondrial extracts. For all immunoblotting, the Odyssey LI-COR scanner and software (LI-COR Biotechnology, Lincoln, NE) were used for detection and quantitative analysis.

Materials. Humanin-G ([Gly14]-Humanin) was obtained from AnaSpec (Fremont, CA). Primary antibodies for STAT3, pSTAT3(Tyr705), pSTAT3(Ser727), and VDAC1 anti-rabbit were obtained from Cell Signaling Technology (Danvers, MA). Primary antibodies for GRIM19, 13-actin, and VDAC1 anti-mouse were obtained from Santa Cruz Biotechnology (Dallas, TX). The Odyssey blocking buffer, LI-COR goat anti-rabbit IR-800, goat anti-mouse IR-680 antibodies, and the 4× Protein Sample Loading Buffer were obtained from LI-COR Biotechnology (Lincoln, NE). The IBind Western System solution kit was purchased from Thermo Fischer Scientific (Waltham, MA). The NuPAGE LDS Sample Buffer, and Western blot gels were purchased from Life Technologies (Grand Island, NY). All other chemicals were obtained from Sigma-Aldrich (St. Louis, MO).

Statistical analysis. Statistical analysis was performed using SigmaPlot for Windows Version 14.5 (Systat Software, San Jose, CA). Data are represented as means±SEM of n=4-22 animals for each group. For multiple group analysis at a single time point, one-way analysis of variance (ANOVA) with Student-Newman-Keuls correction was used. For multiple group analysis at different time points, a two-way ANOVA with Student-Newman-Keuls correction was performed. If data failed to follow a normal distribution, a Mann-Whitney Rank Sum test or an ANOVA on ranks test was performed. The Wilcoxon and log-rank tests were performed for statistical analysis in survival studies (n=7 animals for each group) using JMP®Pro (SAS Institute Inc., Cary, NC). A P value less than 0.05 was considered significant.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating hemorrhagic shock in an individual in need thereof, comprising administering a humanin protein or an analog thereof to said individual.

2. (canceled)

3. The method of claim 1, wherein said humanin protein or analog thereof has at least 90%, or at least 95% sequence homology to a peptide having a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

4. The method of claim 1, wherein said humanin protein or analog thereof is selected from HN, HNG, HNA, AGA-HNG, HN17, HNG17, AGA-C8R-HNG17, colivelin, and combinations thereof.

5. The method of claim 1, wherein said humanin protein or analog thereof is Humanin G (HNG), having the sequence of SEQ ID NO: 2.

6. The method of claim 1, wherein said humanin protein or analog thereof is PEGylated.

7. The method of claim 1, wherein said humanin protein or analog thereof is administered as a pro-drug.

8. (canceled)

9. The method of claim 1 further comprising administering one or both of blood and fluid.

10-16. (canceled)

17. The method of claim 1, wherein said humanin protein or analog thereof is administered for at a period of at least one hour, or at least one day, or at least two days, or at least three days, or at least four days, or at least five days, or at least six days, or at least one week.

18. The method of claim 1, wherein said humanin protein or analog thereof is administered within two hours of admission or diagnosis, within three hours of admission or diagnosis, within four hours of admission or diagnosis, within five hours of admission or diagnosis, or within six hours of admission or diagnosis.

19. The method of claim 1, wherein said humanin protein or analog thereof is administered in an amount of from about 50 μg/kg to 1000 μg/kg, from about 100 μg/kg to about 800 μg/kg, from about 200 μg/kg to about 600 μg/kg, or from about 300 μg/kg to about 500 μg/kg.

20. The method of claim 1, wherein said humanin protein or analog thereof is co-administered with one or both of fluids and blood.

21-23. (canceled)

24. A method of treating an individual for hemorrhagic shock, comprising
a. detecting a biomarker selected from IL-6, IL-10, KC, TNFα, and combinations thereof, and
b. treating said individual having an elevation of said biomarker with humanin protein or an analog thereof to said individual.

25. (canceled)

26. The method of claim 24, wherein said humanin protein or analog thereof has at least 90%, or at least 95% sequence homology to a peptide having a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

27. The method of claim 24, wherein said humanin protein or analog thereof is selected from HN, HNG, HNA, AGA-HNG, HN17, HNG17, AGA-C8R-HNG17, colivelin, and combinations thereof.

28. The method of claim 24, wherein said humanin protein or analog thereof is Humanin G (HNG) (a synthetic mitochondrial peptide having a sequence MAPRGFSCLLLLTGEIDLPVKRRA (SEQ ID NO: 2).

29-31. (canceled)

32. The method of claim 24, further comprising administering one or both of blood and fluid.

33-36. (canceled)

37. The method of claim 24, wherein said humanin protein or analog thereof is administered via intramuscular (IM) delivery, intravenous (IV) delivery, subcutaneous (SC) delivery, intra-arterial delivery, oral delivery, gavage delivery, emollient/skin delivery, transdermal patch, and/or intranasally.

38. The method of claim 24, wherein said humanin protein or analog thereof is administered continuously.

39-45. (canceled)

46. A method for monitoring the progression, severity, and or likelihood of progressing to multiple organ dysfunction syndrome (MODS), comprising detecting serum or blood levels of humanin in an individual at risk for multiple organ dysfunction syndrome.

47. The method of claim 46, comprising administering humanin, or an analog thereof, to an individual determined to have an increased risk of progressing to MODS.
